(19) United States
(12) Patent Application Publication
(10) Pub. No.: US 2023/0263619 A1
(43) Pub. Date: Aug. 24, 2023

(54) MEDICAL DEVICE

(71) Applicant: University College Cardiff Consultants Limited, Cardiff South Glamorgan (GB)

(72) Inventors: Peter Simon Theobald, Cardiff South Glamorgan (GB); Mohamed Mahmoud Elfekky, Cardiff South Glamorgan (GB)

(73) Assignee: University College Cardiff Consultants Limited, Cardiff South Glamorgan (GB)

(21) Appl. No.: 17/769,262

(22) PCT Filed: Oct. 8, 2020
(86) PCT No.: PCT/GB2020/052482

§ 371 Date: Apr. 14, 2022

(30) Foreign Application Priority Data
Oct. 18, 2019 (GB) 1915068.9

(51) Int. Cl. A61F 2/08; A61B 17/06; A61B 90/00

(52) U.S. Cl. CPC A61F 2/0805; A61B 17/06166; A61B 90/06; A61B 2017/06176; A61B 2090/064; A61F 2220/0008

(57) ABSTRACT

The invention concerns a device for use in ligament surgery to locate the best graft attachment site, including parts thereof; a kit of parts for use in ligament surgery; and a method for performing ligament surgery.

### FIELD OF THE INVENTION

The invention concerns a device for use in ligament surgery to locate the optimum graft attachment site, including parts thereof; a kit of parts for use in ligament surgery; and a method for performing ligament surgery.

### BACKGROUND OF THE INVENTION

The anterior cruciate ligament (ACL) is one of a pair of cruciate ligaments (the other being the posterior cruciate ligament) in the human knee. The two ligaments are also called cruciform ligaments, as they are arranged in a crossed formation. The ACL originates from deep within the notch of the distal femur and its proximal fibres fan out along the medial wall of the lateral femoral condyle. There are two bundles of ACL: the anteromedial and the posterolateral, named according to where the bundles insert into the tibial plateau. The tibia plateau is a critical weight-bearing region on the upper extremity of the tibia. The ACL is composed of strong fibrous material and assists in controlling excessive motion; the purpose of the ACL is to resist the motions of anterior tibial translation and internal tibial rotation which is important in order to have rotational stability.

The ACL is one of the four main ligaments of the knee, providing 85% of the restraining force to anterior tibial displacement at 30 degrees and 90 degrees of knee flexion. However, the ACL is the most injured ligament of the four located in the knee joint, with over 100,000 tears occurring annually in the US population alone. Most ACL tears are a result of a non-contact damage such as a sudden change in direction causing the knee to rotate inward; as the knee rotates inward additional strain is placed on the ACL, since the femur and tibia move in opposite directions, causing the ACL to tear. ACL injuries most commonly occur during activities that involve sudden stops or changes in direction, jumping and landing—such as soccer, basketball, football and downhill skiing. Signs and symptoms of an ACL injury usually include: a loud “pop” or a “popping” sensation in the knee; severe pain and an inability to continue activity; rapid swelling; loss of range of motion; and a feeling of instability or “giving way” with weight bearing.

In the case of ACL rupture, almost all cases will require reconstructive surgery on the ACL, during which the torn or ruptured ACL is completely removed and replaced with a piece of tendon or ligament tissue from the patient (autograft) or from a donor (allograft). The two most common sources for this tissue are the patellar ligament and the hamstrings tendon. Commonly the surgeon will drill a hole in the tibia and femur, to form a tibial bone tunnel and femoral bone tunnel, through which the patient's new ACL graft will be guided. Once the graft is pulled through the bone tunnels, the ligament is anchored using screws.

ACL reconstruction aims to restore the normal knee anatomy and kinesiology, through the use of best quality graft tissue, tunnel positioning and a graft fixation that provides sufficient tension for a stable joint and a full range of knee motion during normal flexion and extension. However, with surgery, there is also the possible need for revision surgery. In 10-40% of cases revision surgery is needed due to excessive graft tension, graft damage and graft failure. Most notably, even now, senior surgeons using conventional methods have difficulty identifying the ideal ACL graft attachment position.

It follows that precise graft placement and isometry is critical to achieving the correct knee laxity, whilst poor outcomes are characterised by graft failure, stiff knee, cartilage damage, or graft rupture. The unique anatomy of each patient means achieving the precise graft placement each time is technically demanding. Further it is thought that rigid universal measurements are never accurate to apply, instead dynamic intraoperative real-life measurements are assumed to be the best way to determine tunnel position selection before undertaking graft placement.

In summary, precisely locating the graft is critical to achieving the appropriate isometry (i.e. tension) within the knee and protecting against excessive length changes during the normal function of the knee; because if significant length changes occur with the reconstruction anchored to the bone, the graft will inevitably be stretched, or the fixation will be lost.

Accordingly, we herein disclose a medical device that improves a surgeon's ability to locate the best graft attachment site, ideally, when repairing ligaments.

### STATEMENTS OF INVENTION

According to a first aspect of the invention there is provided a device for use in ligament surgery in a subject comprising:

an elongate member having, at a first end, a manipulating member and, at a second end, a locator plate for determining a graft attachment site, wherein said site locator plate comprises a plurality of holes all of which, or a number of which, have attached thereto or associated therewith an elongate indicator that uniquely identifies each hole with respect to the other holes.

In a preferred embodiment of the invention said elongate indicator is flexible and comprises, most ideally, at least one thread or suture. In this manner, the indicator can be easily bent or manipulated during surgery, such as routing through the joint and drilled tunnels, but has sufficient strength such that under tension it does not stretch and so can be used to determine the tension applied to the indicator.

Reference herein to ligament surgery refers to any surgical procedure requiring correction, repair or replacement of a ligament i.e. the flexible fibrous connective tissue which connects two bones or holds together a joint of the body.

Ideally, the device is used for the reconstruction of the Anterior Cruciate ligament (ACL) of the knee, however as will be appreciated by those skilled in the art, the device can be utilized in the reconstruction of other ligaments of the body, specifically those of joints with a high degree of mobility where there is a need for determining the precise location of graft attachment/isometry.

This includes, but is not limited to, the medial collateral ligament (MCL) of the knee, but also the ligaments of the elbow. Precisely locating a graft attachment site in certain surgery, such as ACL, is critical to achieving the appropriate isometry (i.e. tension) within the joint and protecting against excessive length changes during normal joint functioning.

In yet a further preferred embodiment said locator plate is adapted to mate with an anatomical area to which a ligament graft is to be located and so is specially made, ideally using 3D printing or molding, having regard to the anatomy of the patient. Ideally, the locator plate is bespoke and so manufactured for each patient having regard to the patient's unique anatomy, for example, at the medial wall of the lateral femoral condyle. As will be appreciated by those skilled in the art, visualization of the anatomical shape of the attachment site, and therefore determination of the shape of the locator plate to mate with same, can be determined by numerous means known in the art such as, but not limited to, medical visualization techniques including MRI, X-Ray, CAT or the like.

In a preferred embodiment, said locator plate is substantially circular, elliptical or oval in shape. However, said locator plate can take numerous forms, shapes, sizes and/or geometries and it is selected according to the nature of the anatomical site to which it is to be mated and the ligament surgery to be performed. Preferably, said locator plate has a diameter between 10-25 mm including 0.1 mm therebetween and more ideally still selected from one of the following options 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm.

In a preferred embodiment, said locator plate comprises a plurality of holes, ideally to provide a mesh whereby the risk is minimised of the material of the locator plate covering the best graft site, whilst achieving sufficient rigidity for surgical manipulation. Preferably, between 5-20 holes are provided, more ideally between 6-12 holes. As will be appreciated, the number selected will vary according to the size of the plate being utilized which is chosen accordingly in view of the nature of the ligament repair to be performed.

In yet a further preferred embodiment, said holes have a circular cross-section and have a diameter or cross-sectional size between 1-8 mm including every 0.1 mm therebetween. More ideally still, said holes have a diameter between 1-4 mm, and most ideally between 2-3 mm. In this manner, routine surgical drills can be passed through said holes to mark the anatomical site for subsequent surgery and graft attachment.

In yet a further preferred embodiment still, said holes are spaced apart from one another by an amount of at least 1 mm, and more preferably at least 2 mm. Without wishing to be bound by theory, smaller spacing allows more holes per unit area of plate, which will allow more accurate identification of the best site for graft attachment whilst maintaining structural integrity of the plate.

In yet a further preferred embodiment of the invention, said elongate member is between 5-40 cm in length, and more ideally between 15 −30 cm in length, in each instance including every 1 cm increment there between. As will be appreciated by those skilled in the art, the length of the elongate member will be influenced by the nature of the ligament to be repaired/reconstructed and so varied accordingly.

In yet a further preferred embodiment still, said elongate member further comprises an arcuate member or part that is sized and shaped to accommodate movement of the limb or body part during joint motion whilst the precise ligament site is determined. For example, in the context of ACL repair, the arcuate member or part is positioned adjacent to said locator plate and of a size and shape that allows full movement of the knee. In alternative embodiments, the arcuate member or part is positioned distally from the locator plate according to the nature of the joint and ligament to be repaired.

In a preferred embodiment, said arcuate member or part is between 1-3 cm in length and at its highest point 0.1-3 cm in height, and more preferably between 1.5-2.5 cm in length and at its highest point 0.5-1.5 cm in height. As will be appreciated, the relative dimensions will vary according to the nature of the joint for which ligament repair is required and can be tailored accordingly. Ideally it is positioned near or adjacent the end of said elongate member

In a preferred embodiment of the invention each elongate indicator is indicated by numerous means such as, but not limited to, the provision of a coloured marker associated with each elongate indicator or a different coloured thread attached to or associated with each hole. In this manner, each uniquely coloured thread corresponds to a different hole.

As will be appreciated by those skilled in the art, tension on the elongate indicator or threads during movement can be determined by numerous means, such as commercially available tension measurement devices that measure tension (or movement) when load/force is applied.

Alternatively, and more preferably in the surgical setting, the device according to the invention is used in conjunction with a tension monitor, such as a tube through which the elongate indicator or threads are threaded. In this embodiment, once the locator plate has been secured in position at the potential graft attachment site, the elongate indicator or threads are threaded through the tension monitor and as the joint is moved through its full range of motion the indicator or threads will move in the tension monitor and the indicator or thread which extends least from the tension monitor after movement will be the elongate indicator or thread under the least tension; this elongate indicator or thread will then indicate which hole is to be used to identify the site at which the ligament is to be located.

To facilitate this embodiment, given the dynamics during motion, said elongate indicator or thread is adapted to have uni-directional movement towards the locator plate during movement whereby each elongate indicator or thread passes towards the plate under tension by a graded amount, but reverse movement is prevented. Such uni-directional movement can be achieved by numerous means. In a preferred embodiment, said elongate indicator or thread comprise a plurality of barbs, ideally arranged at least partially around the circumference of same. Additionally, or alternatively, and more preferably, said barbs are arranged longitudinally along at least a part of the length of each elongate indicator or thread, most preferably at least the part retained outside the body in use to avoid snagging during movement, and are spaced with respect to each other along at least a part of the longitudinal axis. In this embodiment, each barbed elongate indicator or thread is provided with its own casing that allows, in combination with said barbs, only unidirectional movement whereby under tension the elongate indicator or thread will be drawn through each casing or tension monitor incrementally and the provision of the barb prevents reverse movement.

In yet a further preferred embodiment of the invention, said elongate indicator or thread may additionally or alternatively comprise a plurality of markings and/or indentations to help monitor the length of same after a selected joint movement such that their length at the end of an assessment can be determined.

In yet a further preferred embodiment, said elongate indicator or thread is manufactured from a bioresorbable material such that, in use, should any thread fragments result from surgery they will be safely absorbable by the body.

It is a preferred feature of the invention that said locator plate, in use, is fixedly attached to the anatomical site. As will be appreciated by those skilled in the art, this can be achieved by numerous means such as, but not limited to, use of a surgical screw to attach the plate to the site. In this embodiment, therefore, the locator plate further comprises an attachment means for attachment of the plate to the anatomical site. This may be in the form of a further hole through which a surgical screw can be threaded.

Additionally, or alternatively, said device further comprises an arm member for securing the device about a limb to be operated upon. In a preferred embodiment, said arm member extends from a first end of the elongation member and terminates at a position near or adjacent to the locator plate.

In yet a further preferred embodiment of the invention said device comprises a locator plate contiguous with said elongated member and so it remains attached thereto throughout the procedure.

Alternatively, and more preferably, said locator plate is releasably attached to said elongate member by an attachment means such as means of a friction fit and/or a screw fix or any other suitable attachment means known to those skilled in the art. Ideally, where a screw fix is used, the plate has an internal thread and said elongated member a complimentary external thread, or vice versa. More preferably, said plate is fixed by a friction fit ideally augmented by lock means such as a Luer lock. In this embodiment, said locator plate can be disposable, or alternatively, sterilized for further use. As will be appreciated, most typically, this embodiment provides a sterilizable and so reusable elongate member and a disposable, single use locator plate.

In a further preferred embodiment of the invention said manipulating region may be fashioned to provide a handle.

In a further preferred embodiment of the invention, said device is fabricated from any appropriate surgical grade material, such as plastic, Polytetrafluoroethylene (PTFE), polyamide, polyethylene or any other appropriate material.

In use, using the manipulating region of the device, the locator plate is introduced into a joint requiring ligament surgery and positioned generally at the anatomical site. More specifically, in the context of ACL, the locator plate is introduced through the lateral arthroscopic portal and secured on the medial wall of the lateral femoral condyle using conventional means. The elongate indicator or thread attached to the holes are retained on the outside of the body, via a pre-drilled tibial hole, and the tension on each indicator or thread is then tested, ideally through a full range of joint motion. The elongate indicator or thread that exhibits the lowest tension through the full range of joint motion indicates, externally to the joint, the locator plate hole nearest to or at the best ligament graft attachment site. The position of the locator plate that yields an elongate indicator or thread under lowest tension during the full range of joint movement is indicative of the best ligament attachment site. Advantageously, because each elongate indicator or thread is uniquely coded, with respect to each locator plate hole, the corresponding hole can be identified and thus the precise tunnel exit point can be determined and used during the attachment phase of the procedure.

Further, in the context of ACL, the knee is moved through the full range of motion to determine the precise location for best femoral tunnel exit point, following which, the surgeon can then prepare the femoral tunnel at the site of the hole determined to have isometry.

According to a second aspect of the invention, there is provided a locator plate as defined herein.

According to a further aspect of the invention there is provided a kit of parts for use in ligament surgery comprising: at least one medical device as herein described and a plurality of locator plates as herein described.

In yet a further preferred kit of the invention still, said kit further comprises at least one tension monitor as herein described.

In the context of ACL surgery, the tension monitor, in the form of a cylindrical tube, is configured to be inserted into the drilled tibia tunnel of the tibia. In this arrangement, once the locator plate has been secured into position at the general graft attachment site, the elongate indicator or threads are inserted into the tension monitor and the movement of same within said tension monitor, during joint movement, is used to determine the elongate indicator or thread under least tension. To facilitate this arrangement, given the dynamics that occur during motion, said elongate indicator or thread is adapted to have uni-directional movement towards the locator plate during movement whereby each thread passes towards the plate under tension by a graded amount, but reverse movement is prevented. In this manner, when the joint is moved each thread moves unidirectionally towards the locator plate and so the length of each thread extending out of the tension monitor diminishes. At the end of joint movement, the longest thread extending from the tension monitor indicates the hole to be used to precisely locate the ligament attachment site.

In a preferred embodiment of the invention said tension monitor is a tube having a diameter between 2-12 mm and more ideally between 5-10 mm. In a further preferred embodiment, said tension member comprises a plurality of channels each configured to receive a single thread. Alternatively, a single channel is provided to receive all threads.

In yet a further preferred embodiment, said tension monitor is longitudinally hinged to move between an open and closed position and in which the elongate indicator(s) or thread(s) are placed, for example, before being inserted into the drilled tibia tunnel of the tibia.

According to a further aspect of the invention there is provided a method for performing ligament surgery in a subject comprising:
a) inserting into a joint to be repaired an elongate member having, at a first end, a locator plate for determining a graft attachment site, wherein said site locator plate comprises a plurality of holes all of which, or a number of which, have attached thereto or associated therewith an elongate indicator that uniquely identifies each hole with respect to the other holes;
b) positioning said locator plate against the anatomical area where the graft is to be located;
c) positioning the elongate indicators outside the joint to be repaired;
d) moving the joint through a single, or range, of motion;
e) determining the elongate indicator under the least tension during step d) and the hole corresponding to said indicator;
f) locating the ligament graft to the anatomical site covered by the hole identified in step e); and
g) removing and/or withdrawing the device from the joint.

In a preferred method of the invention, step c) comprises the use of a tension monitor, such as a cylindrical tube, wherein the elongate indicators are threaded through the tension monitor. In this preferred arrangement, step e) further comprises a determination of the movement of the elongate indicators within said tension monitor during joint movement. More ideally, said elongate indicators are adapted to have uni-directional movement towards the locator plate during movement whereby each elongate indicator passes towards the plate under tension by a different amount and so the length of each elongate indicators extending from the tension monitor, after movement, is indicative of the elongate indicator under least tension and so its hole is the best graft attachment site.

According to yet a further preferred aspect of the invention, there is provided a method for performing ACL ligament surgery in a subject provided with a drilled tibial tunnel comprising:
a) inserting into a knee joint to be repaired an elongate member having, at a first end, a locator plate for determining a graft attachment site, wherein said site locator plate comprises a plurality of holes all of which, or a number of which, have attached thereto or associated therewith an elongate indicator that uniquely identifies each hole with respect to the other holes;
b) positioning said locator plate against the medial wall of the lateral femoral condyle at or approximate to the area where a femoral graft tunnel is to be drilled;
c) threading the elongate indicators through the tibial tunnel and outside the joint to be repaired;
d) moving the knee joint through a single, or range, of motions;
e) determining the elongate indicator under the least tension during step d) and the hole and the hole corresponding to said indicator;
f) drilling the femoral tunnel through the hole identified in step e);
g) removing and/or withdrawing the device from the joint; and
h) attaching the graft to the lateral femoral wall

In a preferred method of the invention, step c) comprises the use of a tension monitor, such as a cylindrical tube, wherein the elongate indicators are threaded through the tension monitor. In this preferred arrangement, step e) further comprises a determination of the movement of the elongate indicators within said tension monitor during joint movement. More ideally, said elongate indicators are adapted to have uni-directional movement towards the locator plate during movement whereby each elongate indicator passes towards the plate under tension by a different amount and so the length of each elongate indicators extending from the tension monitor, after movement, is indicative of the elongate indicator under least tension and so its hole is the best graft attachment site.

It will be apparent to those skilled in the art that the design of the device enables the user to facilitate the correct positioning of femoral tunnel exit point and so graft positioning, improving surgical success and reduces the possibility of post-operative revision.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described by way of example only with reference to the Examples below and to the following Figures wherein:

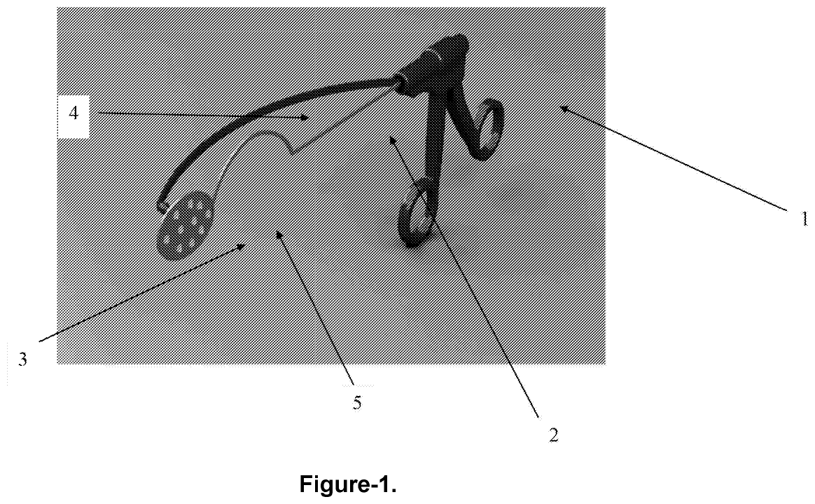

FIG. 1. Shows a perspective view of a medical device in accordance with the invention.

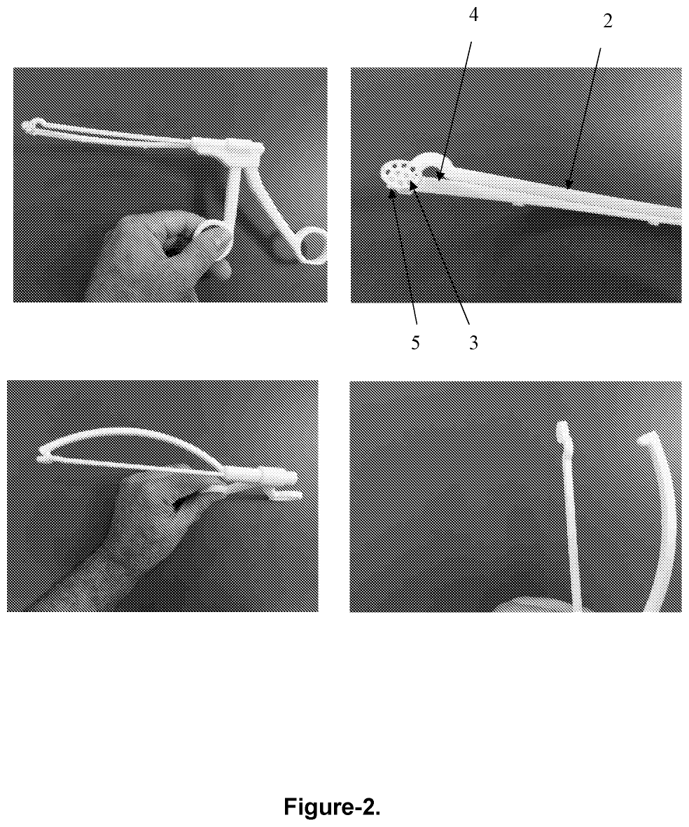

FIG. 2. Shows (I.h.s.) a side elevation and plan view of a medical device in accordance with the invention and (r.h.s.) a side elevation view of a medical device in accordance with the invention and a plan view of a medical device in accordance with the invention when in an open position.

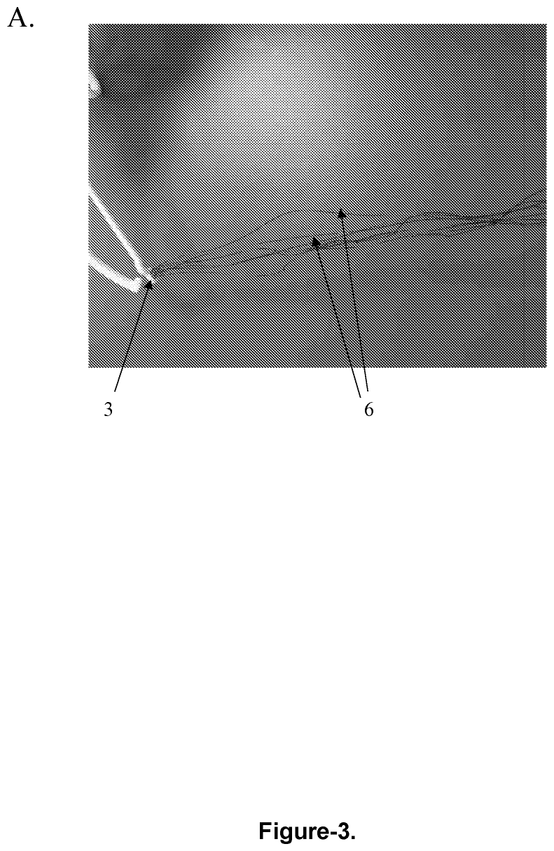

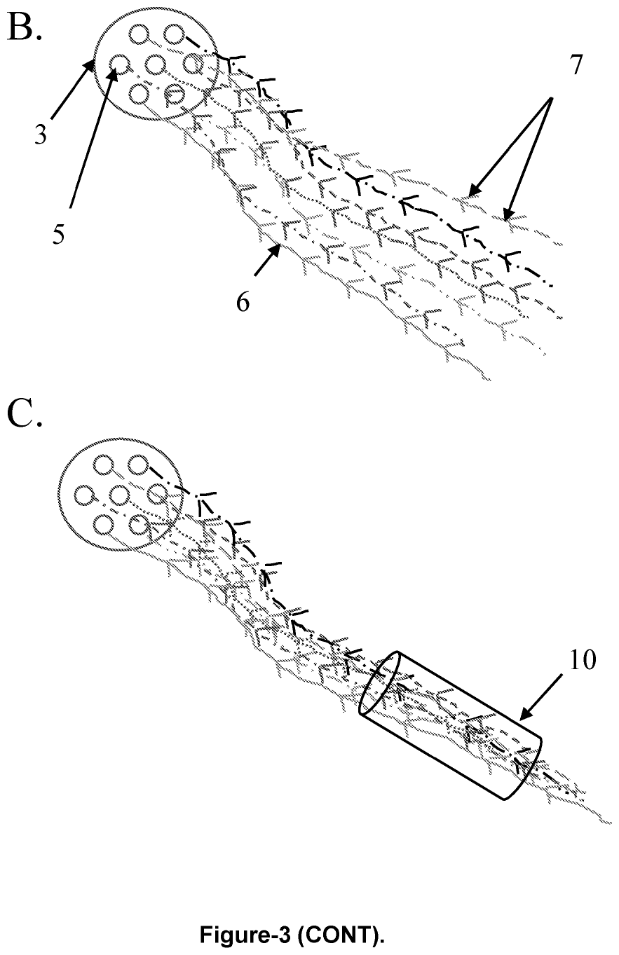

FIG. 3. Shows a perspective view of a tip of the medical device in accordance with the invention.

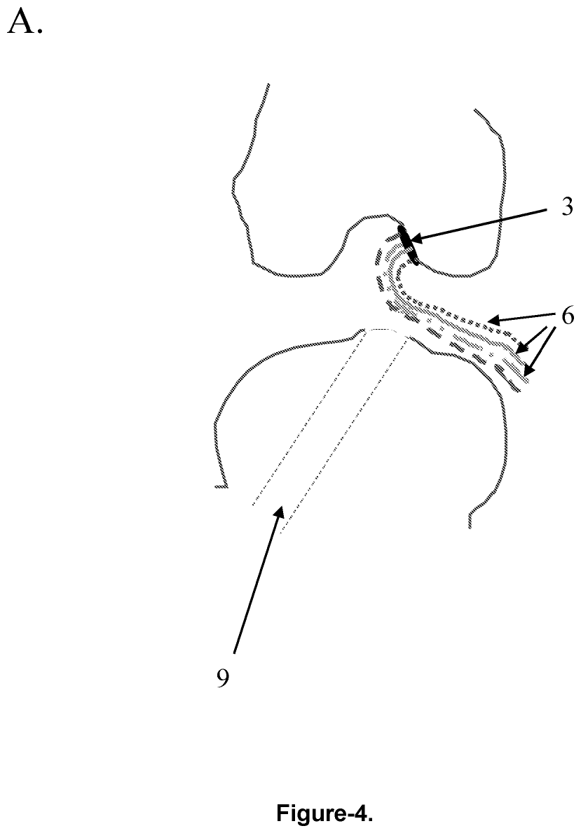

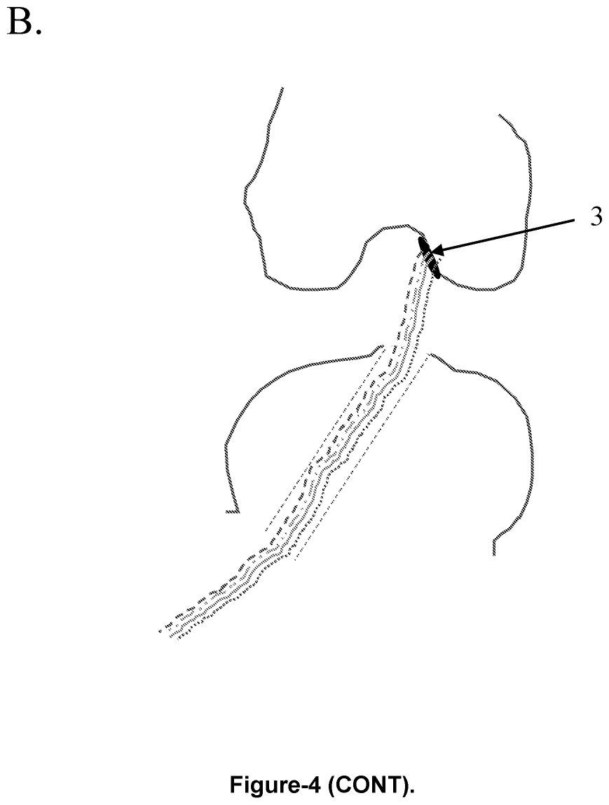

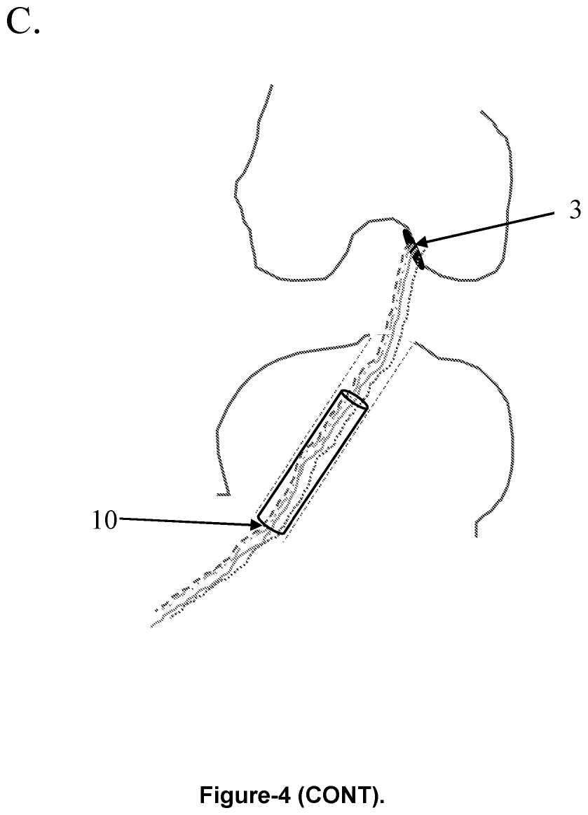

FIG. 4. Shows diagrammatically how the medical device according to the invention is to be used.

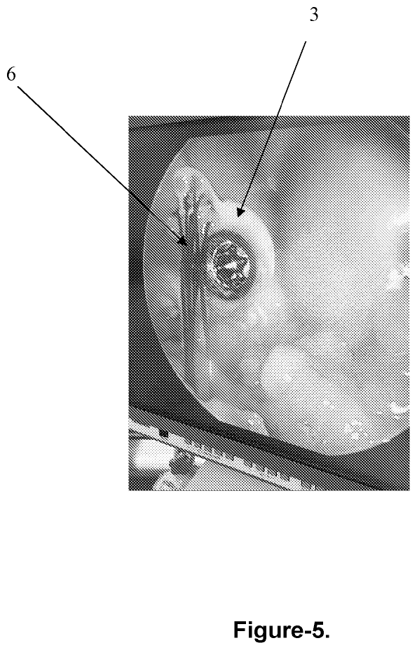

FIG. 5. Surgical insertion and use of the device according to the invention. In this embodiment, the device is fixed to the femoral condyle using a 5 mm cancellous screw. In alternative arrangements (not shown) the device further comprises an arm to generate a pinching force that will clamp the disc in position;

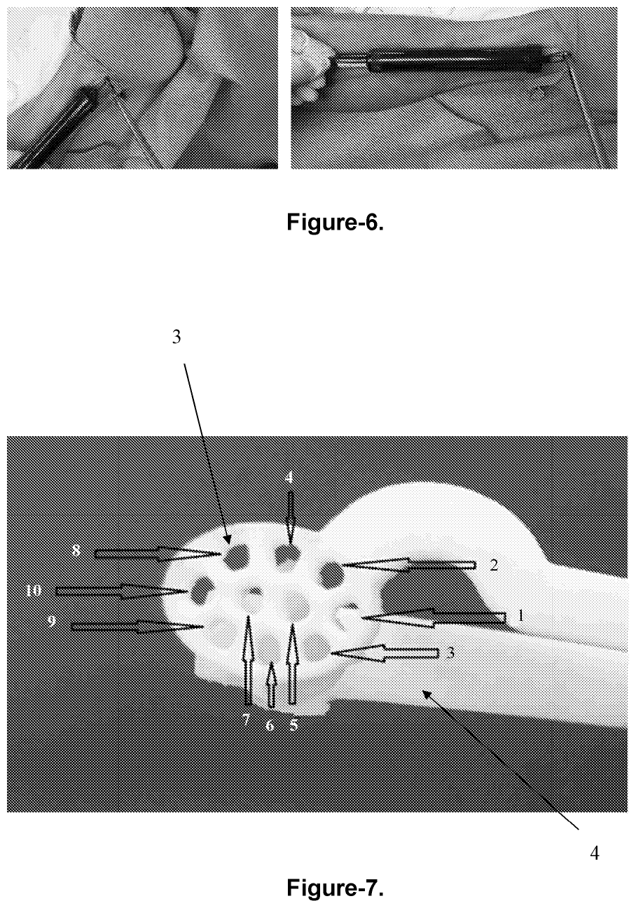

FIG. 6. Shows a suture tension device measures tension on threads through a full range of motion. For proof of concept, the tension device was fixed with a 5 mm cancellous screw to the medial malleolus;

FIG. 7. Shows an example of a tip of a device according the invention with marker holes;

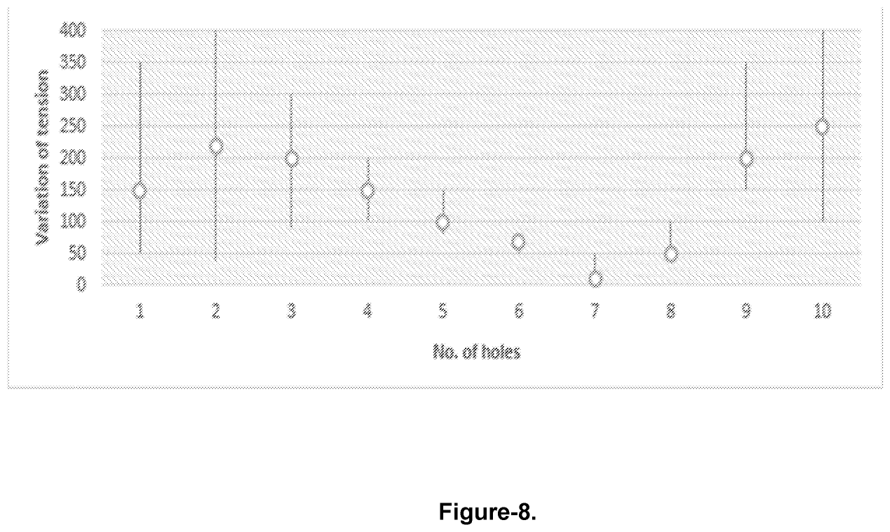

FIG. 8. Shows a graphical representation of the tension measured for each thread and its corresponding hole reference; in this example 7 would be the best ligament attachment position.

Table 1. Table showing the tension measured for each thread and its corresponding hole reference.

Referring now to the figures and, firstly, to FIG. 1 there is shown a perspective view of a medical device in accordance with the invention. It is ideally made from a single moulded or printed material and has a scissor-type handle [1] (although this may vary according to a user's requirements) and originating therefrom is an extension member [2] that terminates in a locator plate [3].

Extension member [2] is typically, though not exclusively, in the form of a fine rod that arcs or bends towards the end nearest the locator plate [3]. This arcing ensure a surgeon can manipulate a joint when the locator plate is in situ without the rod otherwise obstructing movement. Accordingly, the size and shape of the arc may differ for different applications, e.g. elbow would require a device with a smaller sized arc compared to a device for use with a knee joint. The device also includes an optional arm member [4] that originates from handle [1] and runs parallel with extension member [2] but is shaped or curved in a manner so that it can be placed about a limb to engage with same and so steady the device when in use. This feature is preferred in a device where the locator plate [3] is contiguous with the extension member [2] and so remains attached to same throughout use.

FIG. 2 shows a printed version of the device described above. In this device, locator plate [3] is fixed or integral with extension member [2] and formed at the end thereof. Arm member [4] is releasably engageable with extension member [2] under the control of handle [1].

In FIG. 3 there is shown the locator plate [3] and it can be seen that the locator plate includes a series of holes [5], the number and nature of which can be specific to the nature of the joint to be operated upon, e.g. an elbow joint may include a smaller number of smaller holes compared to a locator plate for use with a knee joint. Each one of the holes [5] is threaded with a threadable material [6]. Any type of thread will suffice, however, it is important that each thread distinguishes each threaded hole because the tension in each thread is used to determine which hole represents the best ligament attachment site. Preferably, the thread is resorbable such that, in use, should any thread fragments result from surgery they will be safely absorbable by the body. Any type of indication may be used although it is envisaged that the use of different coloured threads will most easily indicate the hole to be used. This is shown in FIG. 3b, here, each thread is colour coded and, further, provided with a unidirectional movement limiter [7]. Each coloured thread is provided with a number of spaced barbs [7] and in use is inserted in a tension monitor or casing, or tunnel [8] adapted to receive a barbed thread and encase same so that movement is only unidirectional.

Referring now to FIG. 4a, a tibial tunnel [9] is provided through which the distal aspect of the ACL graft is pulled through, to be attached to the medial wall of the tibia. The device is used to insert the locator plate [3] into the knee joint, it can then be held in place during the subsequent manipulations using handle [1] or attached in place using a surgical screw. The coloured coded threads are then threaded through the tibial tunnel (and in any event externalised) (FIG. 4b). With the locator plate [3] in place the joint is manipulated and as it is moved the tension in each thread is measured using a conventional device. The thread under the least tension identifies the hole that represents the precise site for ligament attachment.

To determine the tension of each thread in movement, a tension measuring device (the Salter Brecknell Super Samson, a hand-held, light weighing device with overload protection and tare mechanism) was fixed at its distal end through a screw to the medial malleolus. The proximal end of the tension device was connected to each thread coming from the tibial tunnel and measuring the tension elicited through the full range of motion from full flexion to full extension to test suture near isometricity. Tension force data was retrieved from the tension measuring device.

In one embodiment, the externalised and coloured coded threads are barbed and threaded through a single casing or tension monitor [10] (FIG. 4c). With the locator plate [3] in place, the joint is manipulated and as it is moved the threads are pulled through the casing [10]. The thread that moves the least, and so extends from said casing the furthest, is represent of the thread under least tension and so its corresponding hole represents the precise site for ligament attachment.

In another embodiment, each one of the externalised and coloured coded threads are barbed and threaded through their own casing [10], or single casing each with its own channel. With the locator plate [3] in place the joint is manipulated and, as it is moved, the threads are pulled through their casings [10]. Due to the presence of the barbs, this movement is unidirectional and so at the end of the joint manipulation one of the threads will have moved the least and so extend from its casing the most, this is representative of the thread under least tension and so of the hole that is the precise site for ligament attachment.

FIG. 5 shows an image of a locator plate [3] in place i.e. attached to the general location of a ligament attachment site using a surgical screw and its associated threads.

FIG. 6 shows the device being used on an ankle joint and in combination with a conventional tension measuring device. This shows the distal end of the tension meter, where it is screwed on to the medial malleolus of the ankle joint. The proximal end of the tension meter accepts the sutures that are drawn through the tibial tunnel in the knee

FIG. 7 shows a view of a locator plate [3], in this image the plate is attached to the medical device, although, as shown in FIG. 5, this is not always the case. The locator plate [3] can be shaped for a user's requirements but is most typically circular or cylindrical. It includes a number of holes each one, in use, is threaded for tension measurement. Though not shown, each hole may be coded in a manner that corresponds to the coding used for its thread, this assists with matching each thread with its hole. In this embodiment it can be seen that an arm member [4] is provided for gripping a limb and aiding in the use of the device.

FIG. 8 is a graph that measures tension at each of the locator plate hole potential attachment sites, depicted by holes 1-10 of FIG. 7. It can be seen that the tension in thread 7 corresponding to hole 7 is subject to the least tension during the movement of the joint. Accordingly, this represents the best site for ligament attachment.

Device Designing and 3D Printing

As proof of concept, a device according to the invention was designed with 10 different positioned holes (FIG. 7) for insertion through the lateral arthroscopic portal and fixed with a clamp to the outer lateral femoral cortex to gain stability while moving the knee from flexion to extension for several cycles.

Subject

A disarticulated lower limb human cadaver was used to test a novel mechanical device which was 3D printed. The lower limb cadaver was chosen to be with full range of motion and with the least amount of fat possible to minimize the effects of fatty soft tissue effect on range and ligament tension. The native ACL was severed and a tibial tunnel 9 mm to be drilled in the centre of the tibial footprint of the ACL.

Surgical Technique

Before insertion, proline sutures are connected to the holes in which every thread is passed and tied with a knot at the back side (FIG. 3a). The device with the threads was introduced through the lateral arthroscopic portal (FIG. 5a)

Threads were pulled from a 9-mm tibial tunnel (FIG. 5b) with a suture retrieval handle. In this example, the device was fixed to the femoral condyle via 5 mm cancellous screw to fix it in position (FIG. 5c).

Results:

The data from the tension measuring device testing is shown in table 1 and FIG. 8 using a device with 10 holes enumerated from 1-10 (FIG. 7).

For this study, the assumption was made that we can select a point at which tension in both flexion and extension is the least.

As we can see there were four holes showing minimal tension (5,6,7 and 8). With least tension at hole 7. This was therefore determined to be the position nearest to isometry, and thus the hole can then be used to drill the femoral tunnel for ACL graft attachment.

Discussion:

Anterior cruciate ligament reconstruction is a demanding operation. Tunnel placement is considered one of the crucial determinants in outcome after ACL reconstruction. The aim is to have the graft located at the most isometric point in the position of the original ACL achieving both stability and full range of motion whilst maintaining a length near that of the original ACL. The point of femoral attachment of the graft is crucial and determines graft impingement and whether there will be graft failure.

We herein disclose a simple yet elegant manual device for measuring the best isometric point for each joint to determine optimum graft attachment position.

| | | Full flexion | Flexion of |
|---|---|---|---|
| | Full extension | 160 degrees | 90 Degree |
| No of | net tension | net tension | net tension |
| the hole | in gm | in gm | in gm |
| 1 | 50 | 350 | 150 |
| 2 | 40 | 400 | 220 |
| 3 | 90 | 300 | 200 |
| 4 | 100 | 200 | 150 |
| 5 | 80 | 150 | 100 |
| 6 | 50 | 60 | 70 |
| 7 | 20 | 50 | 10 |
| 8 | 80 | 100 | 50 |
| 9 | 150 | 350 | 200 |
| 10 | 100 | 400 | 250 |

1. A device for use in ligament surgery in a subject comprising:
an elongate member having, at a first end, a manipulating member and, at a second end, a locator plate for determining a graft attachment site, wherein said site locator plate comprises a plurality of holes all of which, or a number of which, have attached thereto or associated therewith an elongate indicator that uniquely identifies each hole with respect to the other holes.

2. The device according to claim 1 wherein said elongate indicator is flexible.

3. The device according to claim 1 wherein said elongate indicator comprises at least one thread or suture.

4. The device according to claim 1 wherein said locator plate is adapted to mate with an anatomical area to which a ligament graft is to be located.

5. (canceled)

6. (canceled)

7. The device according to claim 1 wherein said elongate member further comprises an arcuate member or part that is sized and shaped to accommodate, in use, movement of the limb or body part during joint motion.

8. The device according to claim 7 wherein said arcuate member or part is positioned adjacent, or near, from the end of said elongate member.

9. The device according to claim 1 wherein said elongate indicator is adapted to have uni-directional movement towards the locator plate during movement whereby each elongate indicator passes towards the plate under tension by a graded amount but reverse movement is prevented.

10. The device according to claim 9 wherein said elongate indicator comprise a plurality of barbs arranged circumferentially.

11. The device according to claim 10 wherein said barbs are further arranged longitudinally and are spaced with respect to each other along at least of a part of the longitudinal axis.

12. The device according to claim 1 wherein said elongate indicator comprise a plurality of markings and/or indentations.

13. The device according to claim 1 further comprising an arm member for securing the device about a limb to be operated upon wherein said arm member extends from a first end of the elongation member and terminates at a position near or adjacent to the locator plate.

14. The device according to claim 1 wherein said locator plate is releasably attached to said elongate member by an attachment means.

15. A locator plate comprising a plurality of holes all of which, or a number of which, have attached thereto or associated therewith an elongate indicator that uniquely identifies each hole with respect to the other holes, wherein the locator plate is configured to be part of a ligament surgery device, the ligament surgery device comprising an elongate member having, at a first end, a manipulating member and, at a second end, the locator plate, wherein the locator plate is configured to determine a graft attachment site.

16. The locator plate according to claim 15 wherein said elongate indicator is flexible.

17. The locator plate according to claim 15 wherein said elongate indicator comprises at least one thread or suture.

18. The locator plate according to claim 15 wherein said locator plate is adapted to mate with an anatomical area to which a ligament graft is to be located.

19. (canceled)

20. (canceled)

21. The locator plate according to of claim 15 wherein said elongate indicator is adapted to have uni-directional movement towards the locator plate during movement whereby each thread passes towards the plate under tension by a graded amount but reverse movement is prevented.

22. The locator plate according to claim 21 wherein said elongate indicator comprise a plurality of barbs arranged at least partially circumferentially.

23. The locator plate according to claim 22 wherein said barbs are further arranged longitudinally and are spaced with respect to each other along a part of the longitudinal axis.

24. A kit of parts for use in ligament surgery comprising:
at least one device comprising an elongate member having, at a first end, a manipulating member and, at a second end, a locator plate for determining a graft attachment site, wherein said site locator plate comprises a plurality of holes all of which, or a number of which, have attached thereto or associated therewith an elongate indicator that uniquely identifies each hole with respect to the other holes, and one or more additional locator plates configured to determine a graft attachment site, each additional locator plate comprising a plurality of holes all of which, or a number of which, have attached thereto or associated therewith an elongate indicator that uniquely identifies each hole with respect to the other holes.

25. The kit according to claim 24 further comprising at least one tension monitor.

26. The kit according to claim 25 wherein said tension monitor is a tube whereby, in use, the elongate indicators are inserted into the tension monitor and the movement of the elongate indicators within said tension monitor during joint movement is used to determine the elongate indicator under least tension.

27. (canceled)

28. (canceled)
